(19) United States
(12) Patent Application Publication
(10) Pub. No.: US 2012/0089156 A1
(43) Pub. Date: Apr. 12, 2012

(54) APPARATUS AND METHOD FOR HAIR REMOVAL

(75) Inventors: Giovanni Zangrande, Hong Kong (CN); Elizabeth Jheeta, Hong Kong (CN); Lea Ashfield, Hong Kong (CN)

(73) Assignee: CREATIVE PROPERTIES, LTD.

(21) Appl. No.: 12/901,664

(22) Filed: Oct. 11, 2010

(51) Int. Cl. A61B 17/50

(52) U.S. Cl. 606/133

(57) ABSTRACT

A hair removal apparatus including a first element having a first coupling point and a second coupling point, a second element having a third coupling point and a fourth coupling point, and at least one thread. The first element is configured to allow the first and second coupling points to move relative to each other. The thread includes a first point coupled to the first coupling point, a second point coupled to the second coupling point, a third point coupled to the third coupling point, a fourth point coupled to the fourth coupling point, and a twisted portion.

### FIELD

The present invention relates generally to the field of hair removal devices. More specifically, the invention related to hair removal devices configured to remove hair by capturing the hair in a twisted thread.

### BACKGROUND

Unwanted hair may be removed in a variety of ways. For example, scissors, razors, and other cutting devices remove all or part of the exposed hair while leaving the hair roots in the skin. Hair removed in this manner starts regrowing quickly, requiring frequent grooming to maintain the desired cosmetic effect. Moreover, shaving can result in moderate redness, irritations, cuts, and in-grown hair.

Other methods and apparatuses, including waxes, tweezers, epilators, etc., remove hair including the roots in an action commonly defined as epilation. Hair removed in this manner typically has retarded hair growth providing an effect that is more lasting than cutting or shaving. However, waxing can be a messy process and cause redness and skin irritation persisting for several minutes or hours. Tweezers typically operate on a single hair, or very limited number of hairs at a time and, therefore, are not suitable for application on larger skin areas. Epilating devices incorporating motors are more complex and expensive to manufacture, dependent on proximity or availability of electrical power sources, and result in designs that limit the visibility of the hair to be removed, therefore presenting a less precise and less effective cosmetic action.

### SUMMARY

In one aspect, a hair removal apparatus is provided including a first element having a first coupling point and a second coupling point, a second element having a third coupling point and a fourth coupling point, and at least one thread. The first element is configured to allow the first and second coupling points to move relative to each other. The thread includes a first point coupled to the first coupling point, a second point coupled to the second coupling point, a third point coupled to the third coupling point, a fourth point coupled to the fourth coupling point, and a twisted portion.

In one embodiment, the at least one thread includes a twisted portion, which has a proximal end and a distal end, a first section extending from the proximal end to the first point, a second section extending from the proximal end to the second point, a third section extending from the distal end to the third point, and a fourth section extending from the distal end to the fourth point. In one embodiment, at least one of the coupling points is a hook integrally disposed within an eyelet. In one embodiment, the first element also includes an arm, the first coupling point disposed on the arm; and the first coupling point having a hook, the hook having a first end and a second end, the first end disposed on the arm; and a gap, the gap disposed between the second end of the hook and the arm. In one embodiment, the at least one thread includes a fifth section extending from the third point to a terminal end, the second element includes at least one retaining element disposed on the second element, and the retaining element is configured to retain at least a portion of the fifth section.

In one embodiment, the first element includes a first arm, a handle and the first coupling point disposed on the first arm, a second arm joined to the first arm by a hinge, a second handle and the second coupling point disposed on the second arm. In one embodiment, the first element is configured such that when the first handle moves closer to the second handle, the first coupling point moves away from the second coupling point. In one embodiment, the first handle is disposed between the first coupling point and the hinge. In one embodiment, the first element is configured such that when the first handle moves closer to the second handle, the first coupling point moves closer to the second coupling point. In one embodiment, the hinge is disposed between the first handle and the first coupling point. In one embodiment, the first handle includes a receptacle configured for insertion of one or more fingers.

In another aspect, a hair removal device is provided including a first element having a first coupling point and a second coupling point, a second element disposed on the first element and including a third coupling point, a third element disposed on the first element and including a fourth coupling point, and at least one thread. The first element is configured to allow the first and second coupling points to move relative to each other. The thread includes a first point coupled to the first coupling point, a second point coupled to the second coupling point, a third point coupled to the third coupling point, a fourth point coupled to the fourth coupling point, and a twisted portion.

In one embodiment, the at least one thread includes a twisted portion, which has a proximal end and a distal end, a first section extending from the proximal end to the first point, a second section extending from the proximal end to the second point, a third section extending from the distal end to the third point, and a fourth section extending from the distal end to the fourth point. In one embodiment, at least one of the coupling points is a hook integrally disposed within an eyelet. In one embodiment, the first element also includes an arm, the first coupling point disposed on the arm; and the first coupling point having a hook, the hook having a first end and a second end, the first end disposed on the arm; and a gap, the gap disposed between the second end of the hook and the arm.

In one embodiment, the first element includes a first arm, a handle and the first coupling point disposed on the first arm, a second arm joined to the first arm by a hinge, a second handle and the second coupling point disposed on the second arm. In one embodiment, the first element is configured such that when the first handle moves closer to the second handle, the first coupling point moves away from the second coupling point. In one embodiment, the first handle is disposed between the first coupling point and the hinge. In one embodiment, the first element is configured such that when the first handle moves closer to the second handle, the first coupling point moves closer to the second coupling point. In one embodiment, the hinge is disposed between the first handle and the first coupling point. In one embodiment, the first handle includes a receptacle configured for insertion of one or more fingers.

In another aspect, a method of hair removal is provided. In some embodiments, the method includes providing a hair removal device. In some embodiments, the hair removal device includes a first element having a first coupling point and a second coupling point, a second element having a third coupling point and a fourth coupling point, and at least one thread. In some embodiments, the first element is configured to allow the first and second coupling points to move relative to each other. In some embodiments, the thread includes a first point coupled to the first coupling point, a second point coupled to the second coupling point, a third point coupled to the third coupling point, a fourth point coupled to the fourth coupling point, and a twisted portion. In some embodiments, the method further includes moving the first coupling point relative to the second coupling point to cause tightening of the twisted portion of at least one thread, capturing hair in the twisted portion, and exerting sufficient force to remove the hair from a skin.

Alternative exemplary embodiments relate to other features and combinations of features as may be generally recited in the claims.

### BRIEF DESCRIPTION OF THE DRAWINGS

FIG. 1A is a top view of a hair removal apparatus in an open position, according to an exemplary embodiment.

FIG. 1B is a top view of a hair removal apparatus in a closed position, according to an exemplary embodiment.

FIG. 1C is an enlarged view of an coupling point, according to an exemplary embodiment.

FIG. 1D is an enlarged view of an coupling point, according to an alternate exemplary embodiment.

FIG. 1E is an enlarged view of a passive element, according to an exemplary embodiment.

FIG. 2 is a top view of an active element, according to an alternative exemplary embodiment.

FIG. 3 is a top view of an active element, according to a second alternative exemplary embodiment.

FIG. 4 is a top view of an active element, according to a third alternative exemplary embodiment.

FIG. 5 is a top view of a hair removal apparatus, according to a fourth alternative exemplary embodiment.

FIG. 6 is a top view of a hair removal apparatus, according to a fifth alternative exemplary embodiment.

FIG. 7 is a top view of a hair removal device, according to another exemplary embodiment.

FIGS. 8A-8D are twisted threads, according to alternate exemplary embodiments.

FIGS. 9A and 9B are schematic views illustrating a twisted part of a thread capturing and removing hairs from a skin.

### DETAILED DESCRIPTION

Before turning to the figures, which illustrate several exemplary embodiments in detail, it should be understood that the application is not limited to the details or methodology set forth in the description or illustrated in the figures. It should also be understood that the terminology is for the purpose of description only and should not be regarded as limiting.

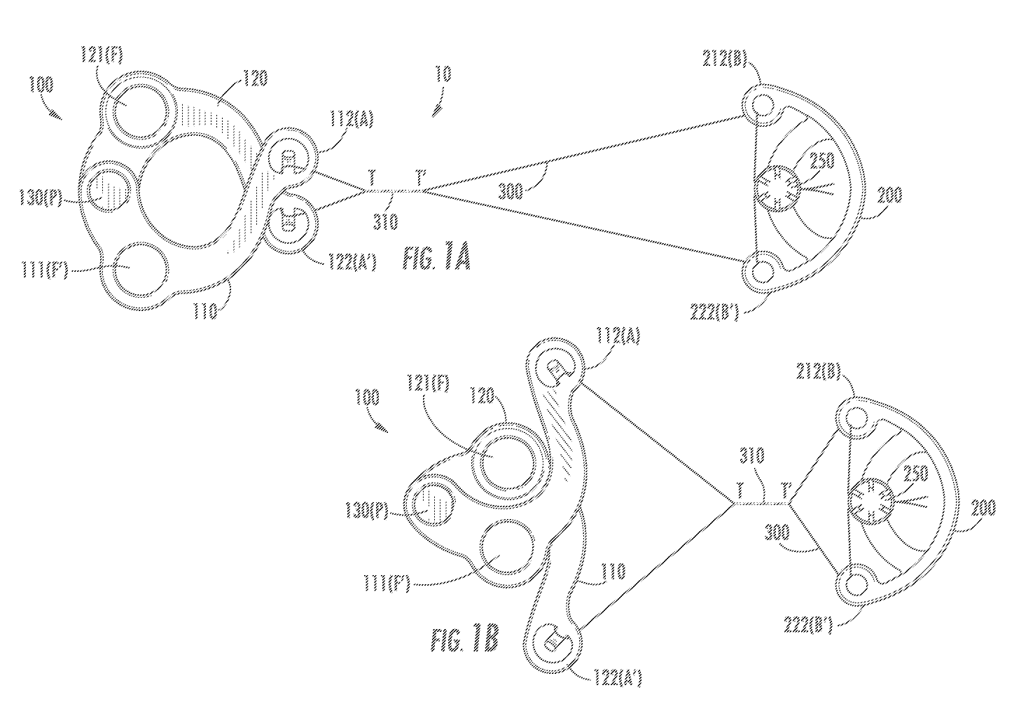

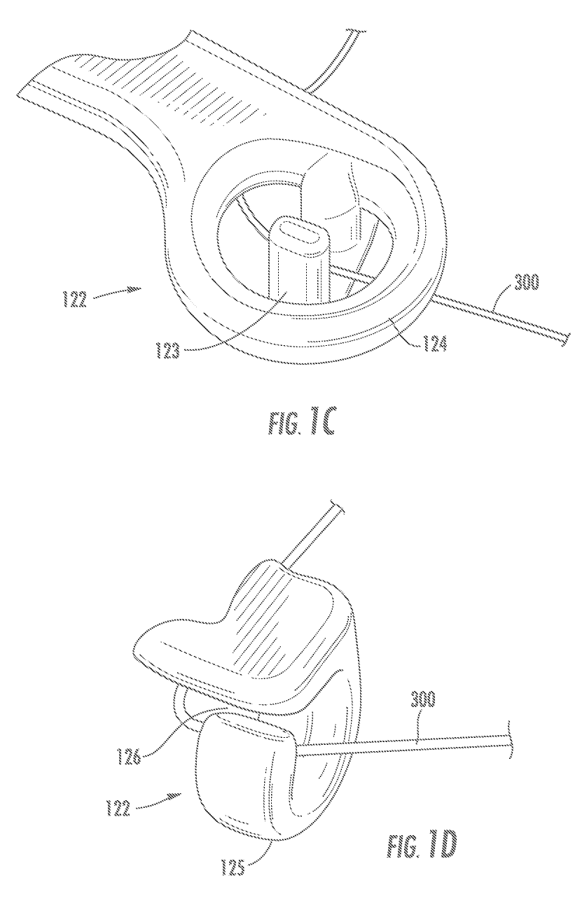

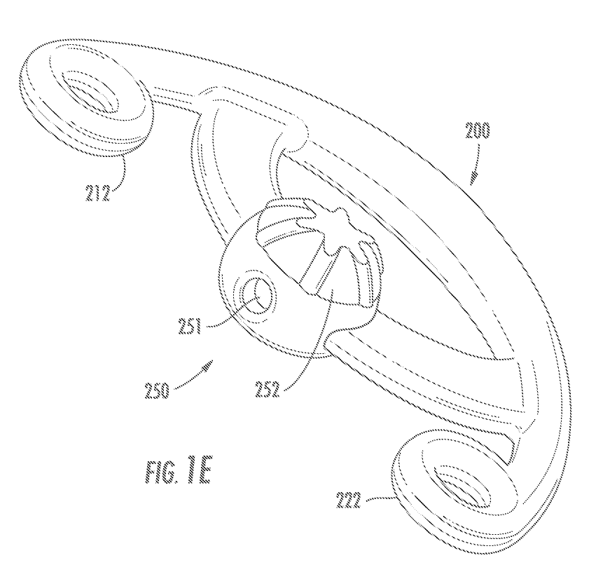

Referring to FIGS. 1A and 1B, a hair removal apparatus, such as threading apparatus 10, is depicted according to an exemplary embodiment. Threading apparatus 10 includes three main elements: a first element, such as active element 100, a second element, such as passive element 200, and at least one thread, such as twisted thread 300.

In the embodiment shown, active element 100 is an independent assembly realized in plastic, metal, or any other rigid material. Active element 100 is shaped to provide a convenient and comfortable gripping surface to allow a user to hold and manipulate threading apparatus 10 during use. Active element 100 includes a first arm and a second arm, such as arms 110 and 120, joined at one end by a hinge, such as pivot 130 (also called P), operating as a fulcrum, around which each arm is capable of limited rotation. Each arm carries at the opposite end from the pivot a first coupling point and a second coupling point, such as coupling points 112 and 122 (also called respectively A and A′).

Referring to FIG. 1C, an enlarged view of coupling point 122 is shown according to an exemplary embodiment. As shown, coupling point 122 includes an example of a hook 123 that is integrally formed within eyelet 124. This arrangement allows thread 300 to slip through eyelet 124 in such a way that thread 300 will be retained by hook 123 with no further action and remain securely lodged in coupling point 122, without being cut or released by the thread movement during the subsequent epilating operation, which will be explained in more detail below.

Referring to FIG. 1D, an enlarged view of coupling point 122 is shown according to an alternate exemplary embodiment. As shown, coupling point 122 includes an example of a hook 125 that is disposed on the arm (shown as second arm 120). Gap 126 is located between an end of hook 125 and the arm. Hook 125 and gap 126 define a perimeter. In one embodiment, the gap is less than one-third of the perimeter. In another embodiment, the gap is less than one-fourth of the perimeter. In another embodiment, the gap is less than one-fifth of the perimeter. In another embodiment, the gap is less than 15 percent of the perimeter. In another embodiment, the gap is less than one-eighth of the perimeter. In another embodiment, the gap is less than one-tenth of the perimeter. In another embodiment, the gap is less than one-twentieth of the perimeter. In another embodiment, the gap is less than five times the diameter of the thread. In another embodiment, the gap is less than four times the diameter of the thread. In another embodiment, the gap is less than three times the diameter of the thread. In another embodiment, the gap is less than two times the diameter of the thread. In another embodiment, the gap is about the diameter of the thread. In another embodiment, the gap is less than the diameter of the thread. In another embodiment, the hook is configured such that the gap is sufficiently small such that the hook performs as an eyelet. This arrangements allow thread 300 to slip into hook 125 in such a way that thread 300 will be retained by hook 125 with no further action and remain securely lodged in coupling point 122, without being cut or released by the thread movement during the subsequent epilating operation, which will be explained in more detail below.

Returning to FIGS. 1A and 1B, in the embodiment shown, each arm 110 and 120 carries between fulcrum and coupling point a receptacle, such as aperture 111, 121 (also called F and F′), suitable for insertion of one or more fingers. As the user opens and closes such fingers, in a scissor-like movement, arms 110 and 120 of active element 100 rotate around pivot 130 so that active element 100 changes between an “open” position (FIG. 1A) and a “closed” position (FIG. 1B). In the embodiment shown, arms 110 and 120 of active element 100 cross over each other so that in the “open” position (FIG. 1A) the distance between apertures FF′ is longer and the angle FPF′ defined between the pivot 130 and the two apertures 111, 121 is wider, while the angle APA′ defined between pivot 130 and each coupling point 112, 122 is narrower. Conversely, in the “closed” position (FIG. 1B) the distance between apertures FF′ is shorter, the angle FPF′ defined between the pivot 130 and the two apertures 111, 112 is narrower, while the angle APA′ defined between pivot 130 and each coupling point 112, 122 is wider.

In the embodiment shown, passive element 200 is an independent assembly realized in plastic, metal or any other rigid material. Passive element 200 is shaped to provide a convenient and comfortable gripping surface to allow a user to hold and manipulate threading apparatus 10 during use. Passive element 200 includes two coupling points 212, 222 (also called respectively B and B′) at opposing ends, so that the distance BB′ is constant and not affected by the operation of active element 100 or thread 300. In this embodiment, coupling points 212 and 222 are realized as eyelets.

Referring to FIG. 1E, an enlarged view of passive element 200 is shown according to an exemplary embodiment. Passive element 200 includes a retaining element, such as thread retaining subassembly 250, which includes holder 251 and rotatable element 252. Terminal ends, for example loose ends, of thread 300 may be inserted in holder 251 and firmly clasped by a 90 degrees rotation of rotatable element 252, obtaining a closed loop. Thread retaining subassembly 250 significantly simplifies the looping of the thread 300.

Returning to FIGS. 1A and 1B, in the embodiment shown, thread 300, made of cotton, silk, polyester, or other material, is connected to four coupling points 112, 122, 212, 222 in such a way to define an X shape, with a twisted portion, such as twisted part 310, residing between coupling points 112, 122, 212, and 222. Twisted part 310 includes proximal and distal ends, such as T and T′.

In the embodiment shown, thread 300 is in the form of a line of thread that is passing through two coupling points 112, 122 on active element 100, each including a hook that is integrally formed within an eyelet, and inside two additional coupling points 212, 222 on passive element 200, each realized as an eyelet. The loose ends of thread 300 are captured and secured in thread retaining subassembly 250, closing the thread loop. The thread loop may subsequently be twisted on itself to form twisted part 310 through various methods including rotating active element 100, rotating passive element 300, or rotating both active element 100 and passive element 300. In one embodiment, the thread loop may be twisted at least ten times. In the embodiment shown, thread 300 represents the only connection between active element 100 and passive element 200 elements, thus eliminating any transmission or support element extending over the surface of skin to be treated, and affording the maximum visibility, accessibility and precise cosmetic action.

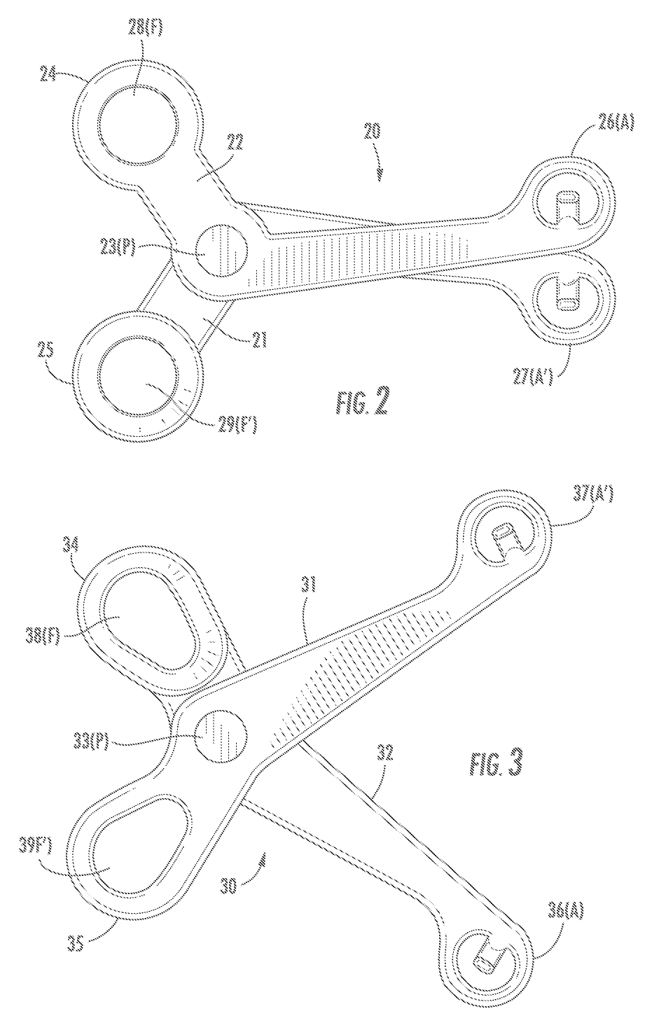

Referring to FIGS. 2-7, several alternative embodiments of the present invention are also possible, utilizing a combination of active and passive elements and twisted threads.

Referring to FIG. 2, active element 20 is depicted according to an alternative exemplary embodiment. Active element 20 includes two arms 21, 22 joined by pivot 23 (also called P) operating as a fulcrum, around which each arm is capable of limited rotation. Each arm 21, 22 carries at one end a handle 24, 25 and at the opposite end an coupling point 26, 27 (also called respectively A and A′). In this embodiment, each handle 24, 25 contains an aperture 28, 29 (also respectively called F and F′) suitable for the insertion of one or more fingers, which may be used to apply force in a scissor-like movement that varies the distance between apertures FF'.

In the embodiment shown, active element 20 is in the “open” position such that the distance between apertures FF′ is longer and angle FPF′ defined between pivot 23 and each aperture 28, 29 is wider, while the angle APA′ defined between pivot 23 and each coupling point 26, 27 is narrower. Conversely, in the “closed” position (not shown, but understood by a person of skill in the art) the distance between apertures FF′ is shorter, angle FPF′ defined between pivot 23 and each apertures 28, 29 is narrower, while angle APA′ defined between the pivot 23 and each coupling point 26, 27 is wider.

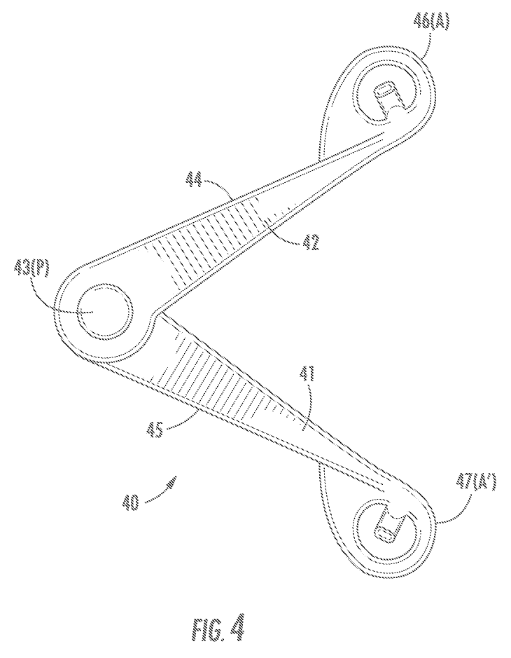

Referring to FIG. 3, active element 30 is depicted according to a second alternative exemplary embodiment. Active element 30 includes two arms 31, 32 joined by pivot 33 (also called P) operating as a fulcrum, around which each arm is capable of limited rotation. Each arm 31, 32 carries at one end a handle 34, 35 and at the opposite end an coupling point 36, 37 (also called respectively A and A′). In this embodiment, each handle 34, 35 contains an aperture 38, 39 (also respectively called F and F′) suitable for the insertion of one or more fingers, which may be used to apply force in a scissor-like movement that varies the distance between apertures FF'.

In the embodiment shown, arms 31 and 32 are crossed at such an angle to achieve an action inverted to the embodiments shown in FIGS. 1 and 2. In the embodiment shown, active element 30 is in the “open” position such that the distance between apertures FF′ is longer and angle FPF′ defined between pivot 33 and each aperture 38, 39 as well as angle APA′ defined between pivot 33 and each coupling point 36, 37 are wider. Conversely, in the “closed” position (not shown, but understood by a person of skill in the art) the distance between apertures FF′ is shorter, and both angle FPF′ defined between pivot 33 and each apertures 38, 39 and angle APA′ defined between the pivot 33 and each coupling point 36, 37 are narrower.

Referring to FIG. 4, active element 40 is depicted according to a third alternative exemplary embodiment. Active element 40 includes two arms 41, 42 joined by pivot 43 (also called P) operating as a fulcrum, around which each arm is capable of limited rotation. Each arm 41, 42 carries an coupling point 46, 47 (also called respectively A and A′) at an opposite end from pivot 43. In the embodiment shown, arms 41 and 42 of active element 40 do not carry apertures suitable for the insertion of one or more fingers, and the force is directly applied to handles, such as outer surfaces 44 and 45 of arms 42 and 41, respectively, by pressure against a spring, in a tweezing-like movement that modifies the angle APA'.

In this embodiment, as understood by a person of skill in the art, arms 41 and 42 may or may not be crossed. Furthermore, as understood by a person of skill in the art, a spring could be a coil spring, torsion spring, or mainspring co-axial to pivot 43, or a coil spring, volute spring, or cantilever spring connected between arms 41 and 42, or any other mechanism to provide a return force. A related embodiment does not provide pivot 43 and forms arms 41 and 42 from a shaped portion of a material with sufficient properties to behave as a spring, for example steel.

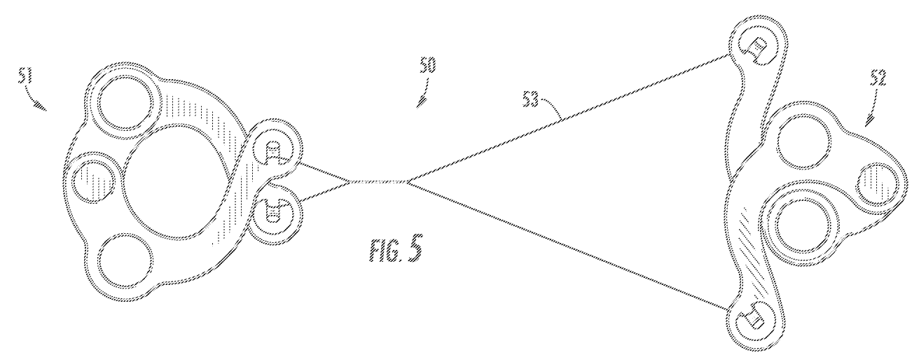

Referring to FIG. 5, hair removal apparatus 50 is depicted according to a fourth alternative exemplary embodiment. Like the preceding exemplary embodiments, hair removal apparatus 50 includes first active element 51 and thread 53. In this embodiment, however, the passive element has been replaced with a second active element 52.

Referring to FIG. 6, hair removal apparatus 60 is depicted according to a fourth alternative exemplary embodiment. Like the preceding exemplary embodiments, hair removal apparatus 60 includes first active element 61 and thread 63. In this embodiment, however, points 64, 65 along thread 63 are held apart by user's hand 62.

In another group of embodiments, a singular passive element may be substituted by two passive elements. Each one of the two passive elements may be made of plastic, metal or other rigid material and can be connected to the active element in such a way to afford an unimpeded line of sight to the area to be treated for hair removal and to allow for unimpeded contact between the thread and such area.

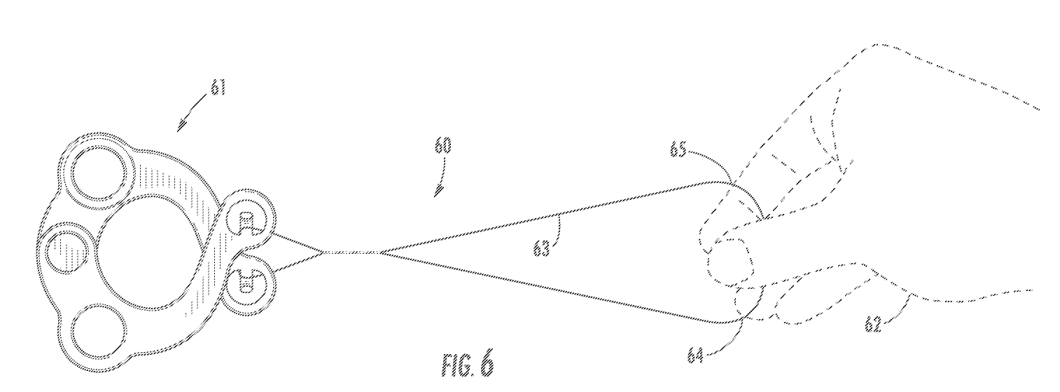

Referring to FIG. 7, hair removal device 70 is depicted according to another exemplary embodiment. In the embodiment shown, two passive elements 72 and 74 are shown in combination with active element 71. This solution is independent from the specific embodiments depicted for the active element.

In all of the above embodiments, coupling points on active and passive element(s) may be alternatively realized as hooks, hooks integrally formed within eyelets, hooks with gaps sufficiently small such that the hooks perform as eyelets, eyelets, or any other retaining element suitable to firmly hold a loop in the thread itself.

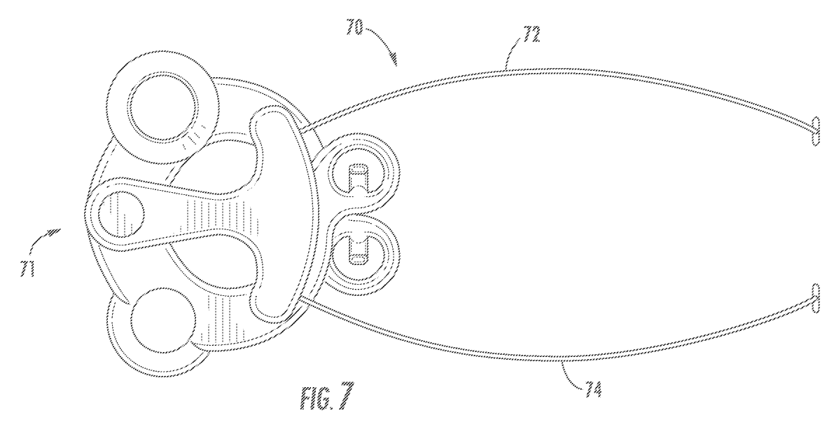

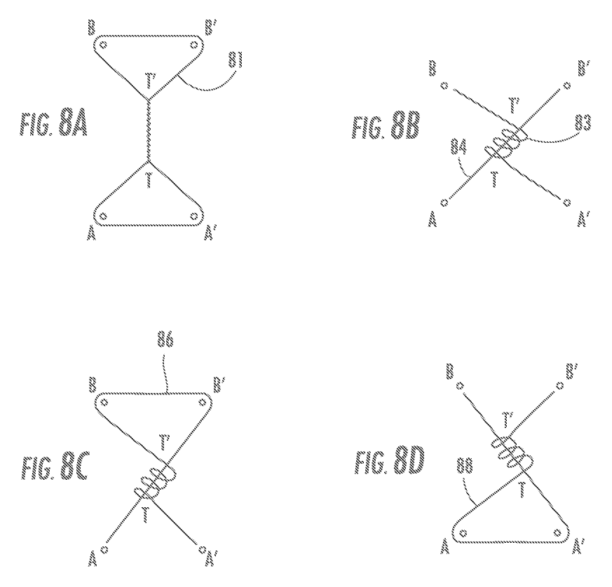

Referring to FIGS. 8A-8D, another group of exemplary embodiments are depicted. In these embodiments, four coupling points are referred to as A, A′, B, and B′. Such embodiments, as well as any alternative way of shaping, fastening, bending, or crossing the thread between the active and passive element are independent from the specific embodiments depicted for the active or passive elements. FIG. 8A depicts thread twisted loop of thread 81 between four coupling points A, A′, B, and B′. FIG. 8B depicts an exemplary embodiment in which a first piece of thread 83 and a second piece of thread 84 are twisted one around the other. First piece of thread 83 is connected between two laterally opposed coupling points: one A′ on the active element, and one B on the passive element. Second piece of thread 84 is connected to points A on the active element and B′ on the passive element. In this embodiment, the coupling points should all be realized with fasteners able to securely hold an open end of the thread—such as, for instance, the retaining subassembly 250 described in the FIG. 2D. FIG. 8C depicts an exemplary embodiment that utilizes a single piece of thread 86 that is connected to a first coupling point A on the active element looped around the two coupling points B, B′ on the passive element, twisted on itself and finally connected to the remaining coupling point A′ on the active element. FIG. 8D depicts an exemplary embodiment that utilizes a single piece of thread 88 that is connected to a first coupling point B on the passive element looped around the two coupling points A, A′ on the active element, twisted on itself and finally connected to the remaining coupling point B′ on the passive element. In this group of embodiments, the coupling points on one element—the passive element in FIG. 8C or the active element in FIG. 8D—may be realized as hooks, as hooks that are integrally formed within eyelets, as hooks with gaps that are sufficiently small such that the hooks perform as eyelets, as eyelets, or any other retaining element suitable to firmly hold a loop in the thread itself, while the coupling points on the opposite element—the active element in FIG. 8C or the passive element in FIG. 8D—should be realized with fasteners able to securely hold an open end of the thread—such as, for instance, the retaining subassembly 250 described in FIG. 2D.

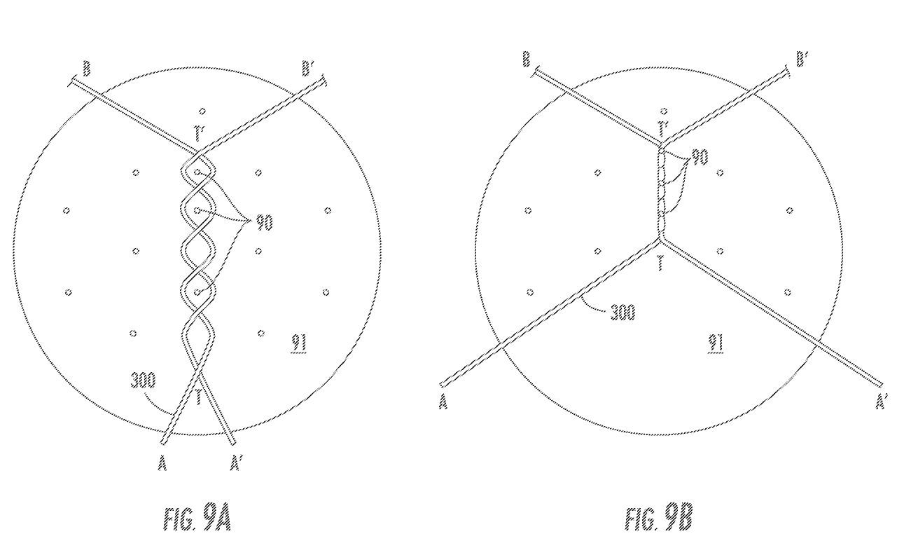

Referring to FIGS. 1 and 9, independently from any specific embodiment, an exemplary description of an operation of hair removal apparatus 10 is the following:

A force exerted on active element 100, which may, for example, be exerted by user's fingers on apertures 111 and 121, forces a increase of angle APA′ defined between each coupling point 112, 122 on active element 100 and pivot 130. Because of the resistance offered by thread 300 and passive element(s) 200 of hair removal apparatus 10, the corresponding angle ATA′ defined between each coupling point 112, 122 on active element 100 and beginning point T of the twisted part 310 (also called TT′) of thread 300 is forced to increase. Referring to FIGS. 9A and 9B, as the angle ATA′ increases, thread 300 becomes tighter, firmly capturing hair 90 in the loops of its twisted part TT′ and such twisted part TT′ moves in the direction of passive element 200, exerting a force that plucks the captured hair 90 by their roots. The reversal of the force on active element 100 causes the apparatus to return in the initial condition, ready for a subsequent action.

In another exemplary use of an exemplary embodiment, the user may, holding active element 100 in one hand with fingers open and passive element 200 in the other hand, move user's hands apart until thread 300 is tensioned and position beginning point T of the twisted part 310 of thread 300 along the direction of the desired hair removal and opposite to the natural growth direction of the hair to be removed. While keeping thread 300 tensioned between two elements 100, 200, one in each hand, close user's fingers holding active element 100. This causes the angle ATA′ defined between each coupling point 112, 122 on active element 100 and beginning point T of the twisted part 310 of thread 300 to increase. As the angle ATA′ increases thread 300 becomes tighter, firmly capturing hair 90 in the loops of its twisted part 310 and such twisted part 310 moves in the direction of passive element 200, exerting a force that plucks captured hair 90 at their follicles. Lift thread 300 from skin 91, open user's fingers holding active element 100 and reposition hair removal apparatus 10 as described above, ready to operate again.

As used herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” etc.) are merely used to describe the orientation of various elements in the FIGURES. It should be noted that the orientation of various elements may differ according to other exemplary embodiments, and that such variations are intended to be encompassed by the present disclosure.

Further modifications and alternative embodiments of various aspects of the invention will be apparent to those skilled in the art in view of this description. Accordingly, this description is to be construed as illustrative only. The construction and arrangements, shown in the various exemplary embodiments, are illustrative only. Any of the features, elements, steps, or components of any of the exemplary embodiments discussed above may be used alone or in combination with any of the features, elements, or components of any of the other embodiments discussed above. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. For example, elements shown as integrally formed may be constructed of multiple parts or elements, the position of elements may be reversed or otherwise varied, and the nature or number of discrete elements or positions may be altered or varied. The order or sequence of any process or method steps may be varied or re-sequenced according to alternative embodiments. Other substitutions, modifications, changes and omissions may also be made in the design, operating conditions and arrangement of the various exemplary embodiments without departing from the scope of the present invention in its broader aspects as defined in the following claims.

What is claimed is:

1. A hair removal apparatus comprising:
a first element comprising a first coupling point and a second coupling point, the first element configured to allow the first and second coupling points to move relative to each other;
a second element comprising a third coupling point and a fourth coupling point; and
at least one thread comprising:
a first point coupled to the first coupling point, a second point coupled to the second coupling point, a third point coupled to the third coupling point, and a fourth point coupled to the fourth coupling point; and
a twisted portion.

2. The apparatus of claim 1, wherein:
the twisted portion further comprises a proximal end and a distal end; and
the at least one thread further comprises:
a first section extending from the proximal end to the first point;
a second section extending from the proximal end to the second point;
a third section extending from the distal end to the third point; and
a fourth section extending from the distal end to the fourth point.

3. The apparatus of claim 1, wherein at least one of the coupling points is a hook integrally disposed within an eyelet.

4. The apparatus of claim 1, wherein:
the first element further comprises:
an arm, the first coupling point disposed on the arm; and
the first coupling point comprises:
a hook;
the hook further comprising a first end and a second end, the first end disposed on the arm; and
a gap, the gap disposed between the second end of the hook and the arm.

5. The apparatus of claim 1, wherein:
the at least one thread further comprises a fifth section extending from the third point to a terminal end;
the second element further comprises at least one retaining element disposed on the second element; and
the retaining element is configured to retain at least a portion of the fifth section.

6. The apparatus of claim 1, wherein the first element further comprises:
a first arm;
a handle disposed on the first arm;
the first coupling point disposed on the first arm;
a second arm joined to the first arm by a hinge;
a second handle disposed on the second arm; and
the second coupling point disposed on the second arm.

7. The apparatus of claim 6, wherein the first element is configured such that when the first handle moves closer to the second handle, the first coupling point moves away from the second coupling point.

8. The apparatus of claim 7, wherein the first handle is disposed between the first coupling point and the hinge.

9. The apparatus of claim 6, wherein the first element is configured such that when the first handle moves closer to the second handle, the first coupling point moves closer to the second coupling point.

10. The apparatus of claim 9, wherein the hinge is disposed between the first handle and the first coupling point.

11. The apparatus of claim 6, wherein the first handle comprises a receptacle configured for insertion of one or more fingers.

12. A hair removal device comprising:
a first element comprising a first coupling point and a second coupling point, the first element configured to allow the first and second coupling points to move relative to each other;
a second element disposed on the first element, the second element comprising a third coupling point;
a third element disposed on the first element, the third element comprising a fourth coupling point; and
at least one thread comprising:
a first point coupled to the first coupling point;
a second point coupled to the second coupling point;
a third point coupled to the third coupling point;
a fourth point coupled to the fourth coupling point; and
a twisted portion.

13. The device of claim 12, wherein the at least one thread further comprises:
a twisted portion comprising a proximal end and a distal end;
a first section extending from the proximal end to the first point;
a second section extending from the proximal end to the second point;
a third section extending from the distal end to the third point; and
a fourth section extending from the distal end to the fourth point.

14. The apparatus of claim 12, wherein at least one of the coupling points is a hook integrally disposed within an eyelet.

15. The apparatus of claim 12, wherein:
the first element further comprises:
an arm, the first coupling point disposed on the arm; and
the first coupling point comprises:
a hook;
the hook further comprising a first end and a second end, the first end disposed on the arm; and
a gap, the gap disposed between the second end of the hook and the arm.

16. The apparatus of claim 12, wherein the first element further comprises:
a first arm;
a handle disposed on the first arm;
the first coupling point disposed on the first arm;
a second arm joined to the first arm by a hinge;
a second handle disposed on the second arm; and
the second coupling point disposed on the second arm.

17. The apparatus of claim 16, wherein the first element is configured such that when the first handle moves closer to the second handle, the first coupling point moves away from the second coupling point.

18. The apparatus of claim 17, wherein the first handle is disposed between the first coupling point and the hinge.

19. The apparatus of claim 16, wherein the first element is configured such that when the first handle moves closer to the second handle, the first coupling point moves closer to the second coupling point.

20. The apparatus of claim 19, wherein the hinge is disposed between the first handle and the first coupling point.

21. The apparatus of claim 16, wherein the first handle comprises a receptacle configured for insertion of one or more fingers.

22. A method of removing hair from skin comprising:
providing a hair removal device comprising:
a first element comprising a first coupling point and a second coupling point, the first element configured to allow the first and second coupling points to move relative to each other,
a second element comprising a third coupling point and a fourth coupling point, and
at least one thread comprising:
a first point coupled to the first coupling point,
a second point coupled to the second coupling point,
a third point coupled to the third coupling point,
a fourth point coupled to the fourth coupling point, and
a twisted portion;
moving the first coupling point relative to the second coupling point to cause tightening of the twisted portion of at least one thread;
capturing hair in the twisted portion; and
exerting sufficient force to remove the hair from a skin.
